(19) United States
(12) Patent Application Publication
(10) Pub. No.: US 2010/0234593 A1
(43) Pub. Date: Sep. 16, 2010

(54) Imidazo[4,5-B]Pyridine-7-Carboxamides 704

(75) Inventors: Per Arvidsson, Sodertalje (SE); Jeremy Burrows, Sodertalje (SE); Peter Soderman, Sodertalje (SE); Ulrika Yngve, Sodertalje (SE)

Correspondence Address: ASTRA ZENECA PHARMACEUTICALS LP;GLOBAL INTELLECTUAL PROPERTY, 1800 CONCORD PIKE, WILMINGTON, DE, 19850-5437, US

(73) Assignee: ASTRAZENECA AB, Sodertalje (SE)

(21) Appl. No.: 12/593,432

(22) PCT Filed: Mar. 28, 2008
(86) PCT No.: PCT/SE08/50356

§ 371 Date: Mar. 30, 2010

Related U.S. Application Data
(60) Provisional application No. 60/909,035, filed on Mar. 30, 2007.

(51) Int. Cl. C07D 413/14; C07D 471/04

(52) U.S. Cl. 544/127; 546/118

(57) ABSTRACT

The present invention relates to new compounds of formula (I) as a free base or a pharmaceutically acceptable salt, solvate or solvate of salt thereof, a process for their preparation and new intermediates used therein, pharmaceutical formulations containing said therapeutically active compounds and to the use of said active compounds in therapy.

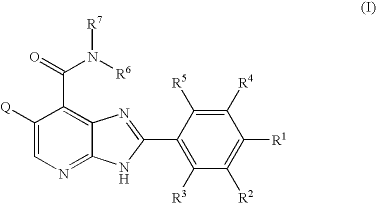

The present invention relates to new compounds of formula I, as a base or a pharmaceutically acceptable salt, solvate or solvate of salt thereof, to pharmaceutical formulations containing said compounds and to the use of said compounds in therapy. The present invention further relates to a process for the preparation of compounds of formula I and to new intermediates used therein.

### BACKGROUND OF THE INVENTION

Glycogen synthase kinase 3 (GSK3) is a serine/threonine protein kinase composed of two isoforms (α and β), which are encoded by distinct genes but are highly homologous within the catalytic domain. GSK3 is highly expressed in the central and peripheral nervous system. GSK3 phosphorylates several substrates including tau, β-catenin, glycogen synthase, pyruvate dehydrogenase and elongation initiation factor 2b (eIF2b). Insulin and growth factors activate protein kinase B, which phosphorylates GSK3 on serine 9 residue and inactivates it.

### Alzheimer's Disease (AD) Dementias, and Taupathies.

AD is characterized by cognitive decline, cholinergic dysfunction and neuronal death, neurofibrillary tangles and senile plaques consisting of amyloid-β deposits. The sequence of these events in AD is unclear, but is believed to be related. Glycogen synthase kinase 3β (GSK3β) or Tau phosphorylating kinase selectively phosphorylates the microtubule associated protein Tau in neurons at sites that are hyperphosphorylated in AD brains. Hyperphosphorylated tau has lower affinity for microtubules and accumulates as paired helical filaments, which are the main components that constitute neurofibrillary tangles and neuropil threads in AD brains. This results in depolymerization of microtubules, which leads to dying back of axons and neuritic dystrophy. Neurofibrillary tangles are consistently found in diseases such as AD, amyotrophic lateral sclerosis, parkinsonism-dementia of Gaum, corticobasal degeneration, dementia pugilistica and head trauma, Down's syndrome, postencephalatic parkinsonism, progressive supranuclear palsy, Niemann-Pick's Disease and Pick's Disease. Addition of amyloid-β to primary hippocampal cultures results in hyperphosphorylation of tau and a paired helical filaments-like state via induction of GSK3β activity, followed by disruption of axonal transport and neuronal death (Imahori and Uchida, J. Biochem. 1997, 121:179-188). GSK3β preferentially labels neurofibrillary tangles and has been shown to be active in pre-tangle neurons in AD brains. GSK3 protein levels are also increased by 50% in brain tissue from AD patients. Furthermore, GSK3β phosphorylates pyruvate dehydrogenase, a key enzyme in the glycolytic pathway and prevents the conversion of pyruvate to acetyl-Co-A (Hoshi et al., PNAS 1996, 93: 2719-2723). Acetyl-Co-A is critical for the synthesis of acetylcholine, a neurotransmitter with cognitive functions. Accumulation of amyloid-β is an early event in AD. GSK Tg mice show increased levels of amyloid-β in brain. Also, PDAPP mice fed with Lithium show decreased amyloid-β levels in hippocampus and decreased amyloid plaque area (Su et al., Biochemistry 2004, 43: 6899-6908). Thus, GSK3β inhibition may have beneficial effects in progression as well as the cognitive deficits associated with Alzheimer's disease and other above-referred to diseases.

### Chronic and Acute Neurodegenerative Diseases

Growth factor mediated activation of the PI3K/Akt pathway has been shown to play a key role in neuronal survival. The activation of this pathway results in GSK3β inhibition. Recent studies (Bhat et. al., PNAS 2000, 97: 11074-11079) indicate that GSK3β activity is increased in cellular and animal models of neurodegeneration such as cerebral ischemia or after growth factor deprivation. For example, the active site phosphorylation was increased in neurons vulnerable to apoptosis, a type of cell death commonly thought to occur in chronic and acute degenerative diseases such as cognitive disorders, Alzheimer's Disease, Parkinson's Disease, amyotrophic lateral sclerosis, Huntington's Disease and HIV dementia and traumatic brain injury; and as in ischemic stroke. Lithium was neuroprotective in inhibiting apoptosis in cells and in the brain at doses that resulted in the inhibition of GSK3β. Thus GSK3β inhibitors could be useful in attenuating the course of neurodegenerative diseases.

### Bipolar Disorders (BD)

Bipolar Disorders are characterised by manic episodes and depressive episodes. Lithium has been used to treat BD based on its mood stabilising effects. The disadvantage of lithium is the narrow therapeutic window and the danger of overdosing that can lead to lithium intoxication. The discovery that lithium inhibits GSK3 at therapeutic concentrations has raised the possibility that this enzyme represents a key target of lithium's action in the brain (Stambolic et al., Curr. Biol. 1996, 68(12):1664-1668, 1996; Klein and Melton; PNAS 1996, 93:8455-8459; Gould et al., Neuropsychopharmacology, 2005, 30:1223-1237). GSK3 inhibitor has been shown to reduce immobilisation time in forced swim test, a model to assess on depressive behavior (O'Brien et al., J Neurosci 2004, 24(30): 6791-6798). GSK3 has been associated with a polymorphism found in bipolar II disorder (Szczepankiewicz et al., Neuropsychobiology. 2006, 53: 51-56). Inhibition of GSK3β may therefore be of therapeutic relevance in the treatment of BD as well as in AD patients that have affective disorders.

### Schizophrenia

Accumulating evidence implicates abnormal activity of GSK3 in mood disorders and schizophrenia. GSK3 is involved in signal transduction cascades of multiple cellular processes, particularly during neural development. (Kozlovsky et al., Am. J. Psychiatry, 2000, 157, 5: 831-833) found that GSK3β levels were 41% lower in the schizophrenic patients than in comparison subjects. This study indicates that schizophrenia involves neurodevelopmental pathology and that abnormal GSK3 regulation could play a role in schizophrenia. Furthermore, reduced β-catenin levels have been reported in patients exhibiting schizophrenia (Cotter et al., Neuroreport 1998, 9(7):1379-1383). Atypical antipsychotics such as olanzapine, clozapine, quetiapine, and ziprasidone, inhibits GSK3 by increasing ser9 phosphorylation suggesting that antipsychotics may exert their beneficial effects via GSK3 inhibition (Li X. et al., Int. J. of Neuropsychopharmacol, 2007, 10: 7-19, Epubl. 2006, May 4).

### Diabetes

Insulin stimulates glycogen synthesis in skeletal muscles via the dephosphorylation and thus activation of glycogen synthase. Under resting conditions, GSK3 phosphorylates and inactivates glycogen synthase via dephosphorylation. GSK3 is also over-expressed in muscles from Type II diabetic patients (Nikoulina et al., Diabetes 2000 February; 49(2): 263-71) Inhibition of GSK3 increases the activity of glycogen synthase thereby decreasing glucose levels by its conversion to glycogen. In animal models of diabetes, GSK3 inhibitors lowered plasma glucose levels up to 50% (Cline et al., Diabetes, 2002, 51: 2903-2910; Ring et al., Diabetes 2003, 52: 588-595). GSK3 inhibition may therefore be of therapeutic relevance in the treatment of Type I and Type II diabetes and diabetic neuropathy.

### Alopecia

GSK3 phosphorylates and degrades β-catenin. β-catenin is an effector of the pathway for keratonin synthesis. β-catenin stabilisation may be lead to increase hair development. Mice expressing a stabilised β-catenin by mutation of sites phosphorylated by GSK3 undergo a process resembling de novo hair morphogenesis (Gat et al., Cell, 1998, 95(5): 605-14)). The new follicles formed sebaceous glands and dermal papilla, normally established only in embryogenesis. Thus GSK3 inhibition may offer treatment for baldness.

### Inflammatory Disease

The discovery that GSK3 inhibitors provide anti-inflammatory effects has raised the possibility of using GSK3 inhibitors for therapeutic intervention in inflammatory diseases. (Martin et al., Nat. Immunol. 2005, 6(8): 777-784; Jope et al., Neurochem. Res. 2006, DOI 10.1007/s11064-006-9128-5)). Inflammation is a common feature of a broad range of conditions including Alzheimer's Disease and mood disorders.

### Cancer

GSK3 is overexpressed in ovarian, breast and prostate cancer cells and recent data suggests that GSK3b may have a role in contributing to cell proliferation and survival pathways in several solid tumor types. GSK3 plays an important role in several signal transduction systems which influence cell proliferation and survival such as WNT, PI3 Kinase and NFkB. GSK3b deficient MEFs indicate a crucial role in cell survival mediated NFkB pathway (Ougolkov A V and Billadeau D D. Future Oncol. 2006 February; 2(1):91-100.). Thus, GSK3 inhibitors may inhibit growth and survival of solid tumors, including pancreatic, colon and prostate cancer.

### Bone-Related Disorders and Conditions

GSK3 inhibitors could be used for treatment of bone-related disorders or other conditions, which involves a need for new and increased bone formation. Remodeling of the skeleton is a continuous process, controlled by systemic hormones such as parathyroid hormone (PTH), local factors (e.g. prostaglandin E₂), cytokines and other biologically active substances. Two cell types are of key importance: osteoblasts (responsible for bone formation) and osteoclasts (responsible for bone resorption). Via the RANK, RANK ligand and osteoprotegerin regulatory system these two cell types interact to maintain normal bone turnover (Bell N H, Current Drug Targets—Immune, Endocrine & Metabolic Disorders, 2001, 1:93-102).

Osteoporosis is a skeletal disorder in which low bone mass and deterioration of bone microarchitecture lead to increased bone fragility and fracture risk. To treat osteoporosis, the two main strategies are to either inhibit bone resorption or to stimulate bone formation. The majority of drugs currently on the market for the treatment of osteoporosis act to increase bone mass by inhibiting osteoclastic bone resorption. It is recognized that a drug with the capacity to increase bone formation would be of great value in the treatment of osteoporosis as well as having the potential to enhance fracture healing in patients.

Recent in vitro studies suggest a role of GSK3β in osteoblast differentiation. First, it has been shown that glucocorticoids inhibit cell cycle progression during osteoblast differentiation in culture. The mechanism behind this is activation of GSK3β in osteoblasts, resulting in c-Myc down-regulation and impediment of the G₁/S cell cycle transition. The attenuated cell cycle and reduced c-Myc level are returned to normal when GSK3β is inhibited using lithium chloride (Smith et al., J. Biol. Chem., 2002, 277: 18191-18197). Secondly, inhibition of GSK3β in the pluripotent mesenchymal cell line C3H10T1/2 leads to a significant increase in endogenous β-catenin signaling activity. This, in turn, induces expression of alkaline phosphatase mRNA and protein, a marker of early osteoblast differentiation (Bain et al., Biochem. Biophys. Res. Commun., 2003, 301: 84-91).

### DISCLOSURE OF THE INVENTION

The object of the present invention is to provide compounds having a selective inhibiting effect at GSK3. Accordingly, the present invention provides a compound of the formula I:

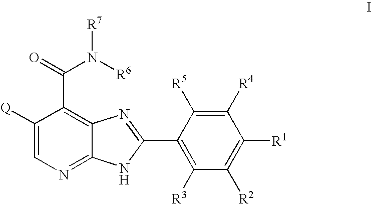

wherein;
Q is halogen;

### R¹ is CH₂NR^{b}R^{c};

R², R³, R⁴ and R⁵ are independently selected from hydrogen and C₁₋₃alkyl;
R⁶ is hydrogen or C₁₋₆alkyl;
R⁷ is selected from hydrogen, C₁₋₆alkyl, C₁₋₆alkylaryl, aryl and heteroaryl, said C₁₋₆alkyl, C₁₋₆alkylaryl, aryl and heteroaryl are optionally substituted with one or more A;
A is halo, CN, OR^{a} or NR^{b}R^{c};
R^{a} is hydrogen, C₁₋₃alkyl or C₁₋₃haloalkyl, said C₁₋₃alkyl or C₁₋₃haloalkyl is optionally substituted with one or more C₁₋₃alkoxy;
R^{b} and R^{c} may, together with the atom to which they are attached, form a 4-, 5- or 6-membered heterocyclic ring containing one or more heteroatoms selected from N, O or S, wherein said heterocyclic ring is optionally substituted with one or more halo, C₁₋₃alkyl or C₁₋₃haloalkyl, and in which any sulphur atom is optionally oxidised to —SO₂—;
as a base or a pharmaceutically acceptable salt, solvate or solvate of a salt thereof.

In another aspect of the invention, there is provided compounds of formula I, wherein Q is halogen, said halogen being selected from bromo and chloro.

In another aspect of the invention, there is provided compounds of formula I, wherein R¹ is CH₂NR^{b}R^{c}, said R^{b}R^{c} together with the atom to which they are attached, form a 6-membered heterocyclic ring containing one or more heteroatoms selected from N or O.

In another aspect of the invention, there is provided compounds of formula I, wherein said R², R³, R⁴ and R⁵ are hydrogen.

In another aspect of the invention, there is provided compounds of formula I, wherein R⁶ and R⁷ are independently selected from hydrogen and C₁₋₆alkyl, said C₁₋₆alkyl being optionally substituted with one or more A. In one embodiment of this aspect, said C₁₋₆alkyl is substituted with one A, said A being OR^{a}. In another embodiment of this aspect, said C₁₋₆alkyl is substituted with one A, said A being OR^{a}, and said R^{a} in OR^{a} represents C₁₋₃alkyl.

In another aspect of the invention, there is provided compounds of formula I, wherein Q is halogen; R¹ is CH₂NR^{b}R^{c}, R^{b}R^{c} together with the atom to which they are attached, form a 4-, 5- or 6-membered heterocyclic ring containing one or more heteroatoms selected from N, O or S; R², R³, R⁴ and R⁵ are hydrogen; R⁶ is hydrogen and R⁷ is C₁₋₆alkyl, said C₁₋₆alkyl is substituted with one A, said A being OR^{a}, said R^{a} in OR^{a} representing C₁₋₃alkyl.

In another aspect of the invention, there is provided compounds of formula I, wherein Q is halogen; R¹ is CH₂NR^{b}R^{c}, R^{b}R^{c} together with the atom to which they are attached, form a 6-membered heterocyclic ring containing one or more heteroatoms selected from N, O or S; R², R³, R⁴ and R⁵ are hydrogen; R⁶ is hydrogen and R⁷ is C₁₋₆alkyl, said C₁₋₆alkyl is substituted with one A, said A being OR^{a}, wherein said R^{a} is C₁₋₃alkyl.

In another aspect of the invention, there is provided compounds of formula I, wherein Q is halogen; R¹ is CH₂NR^{b}R^{c}, R^{b}R^{c} together with the atom to which they are attached, form a morpholine; R², R³, R⁴ and R⁵ are hydrogen; R⁶ is hydrogen and R⁷ is C₁₋₆alkyl, said C₁₋₆alkyl is substituted with one A, said A being OR^{a}, wherein said R^{a} is C₁₋₃alkyl.

In another aspect of the invention, there is provided compounds of formula I, said compounds being selected from:
6-Bromo-N-(3-methoxypropyl)-2-[4-(morpholin-4-ylmethyl)phenyl]-3H-imidazo[4,5-b]pyridine-7-carboxamide hydrochloride; and
6-Chloro-N-(3-methoxypropyl)-2-[4-(morpholin-4-ylmethyl)phenyl]-3H-imidazo[4,5-b]pyridine-7-carboxamide hydrochloride;
6-Fluoro-N-(3-methoxypropyl)-2-[4-(morpholin-4-ylmethyl)phenyl]-3H-imidazo[4,5-b]pyridine-7-carboxamide;
   as a base or alternative pharmaceutically acceptable salt, solvate or solvate of a salt thereof.

In another aspect of the invention, there is provided a compound selected from:
Methyl 4-(6-bromo-3H-imidazo[4,5-b]pyridin-2-yl)benzoate;
Methyl 4-(6-chloro-3H-imidazo[4,5-b]pyridin-2-yl)benzoate;
Methyl 4-(6-bromo-7-chloro-3H-imidazo[4,5-b]pyridin-2-yl)benzoate;
Methyl 4-(6,7-dichloro-3H-imidazo[4,5-b]pyridin-2-yl)benzoate;
Methyl 4-(6-bromo-7-iodo-3H-imidazo[4,5-b]pyridin-2-yl)benzoate;
Methyl 4-(6-chloro-7-iodo-3H-imidazo[4,5-b]pyridin-2-yl)benzoate;
4-(6-Bromo-7-iodo-3H-imidazo[4,5-b]pyridin-2-yl)benzoic acid;
4-(6-Chloro-7-iodo-3H-imidazo[4,5-b]pyridin-2-yl)benzoic acid;
6-Bromo-7-iodo-2-[4-(morpholin-4-ylcarbonyl)phenyl]-3H-imidazo[4,5-b]pyridine;
6-Chloro-7-iodo-2-[4-(morpholin-4-ylcarbonyl)phenyl]-3H-imidazo[4,5-b]pyridine;
6-Bromo-7-iodo-2-[4-(morpholin-4-ylmethyl)phenyl]-3H-imidazo[4,5-b]pyridine;
6-Chloro-7-iodo-2-[4-(morpholin-4-ylmethyl)phenyl]-3H-imidazo[4,5-b]pyridine;
6-Bromo-7-iodo-2-[4-(morpholin-4-ylmethyl)phenyl]-3H-imidazo[4,5-b]pyridine; and
6-Chloro-7-iodo-2-[4-(morpholin-4-ylmethyl)phenyl]-3H-imidazo[4,5-b]pyridine.

In one embodiment of said aspect, there is provided use of said compound(s) as an intermediate(s) in the process of preparing a compound according to formula I.

Listed below are definitions of various terms used in the specification and claims to describe the present invention.

In this specification the term “alkyl” includes both straight and branched chain as well as cyclic alkyl groups. The term C₁₋₃alkyl having 1 to 3 carbon atoms and may be, but is not limited to, methyl, ethyl, n-propyl, i-propyl, or cyclopropyl. The term C₁₋₆alkyl having 1 to 6 carbon atoms and may be, but is not limited to, methyl, ethyl, n-propyl, i-propyl, n-butyl, i-butyl, s-butyl, t-butyl, n-pentyl, i-pentyl, t-pentyl, neo-pentyl, n-hexyl, i-hexyl, cyclopentyl or cyclohexyl.

The term “C₁₋₃alkoxy” includes both straight and branched chains. The term “C₁₋₃alkoxy” having 1 to 3 carbon atoms and may be, but is not limited to, methoxy, ethoxy, n-propoxy, or i-propoxy.

The term “halo” or “halogen” refers to fluorine, chlorine, bromine and iodine.

The term “haloalkyl” refers to an alkyl group, defined as above, in which one or several of the hydrogen substituents have been replaced by halogen substituents, in which the term halogen is defined as above.

The term “aryl” refers to an optionally substituted monocyclic or bicyclic hydrocarbon ring system containing at least one unsaturated aromatic ring. The “aryl” may be fused with a C₅₋₇cycloalkyl ring to form a bicyclic hydrocarbon ring system. Examples and suitable values of the term “aryl”, but not limiting, are phenyl, naphthyl, indanyl or tetralinyl. The term “C₁₋₆alkylaryl”, includes both substituted and unsubstituted alkylaryl groups, which may be substituted on the alkyl and/or the aryl and may be, but are not limited to benzyl, methylphenyl or ethylphenyl.

As used herein, “heteroaryl” refers to an aromatic heterocycle having at least one heteroatom ring member such as sulfur, oxygen, or nitrogen. Heteroaryl groups include monocyclic and polycyclic (e.g., having 2, 3 or 4 fused rings) systems. Examples of heteroaryl groups include without limitation, pyridyl (i.e., pyridinyl), pyrimidinyl, pyrazinyl, pyridazinyl, triazinyl, furyl (i.e. furanyl), quinolyl, isoquinolyl, thienyl, imidazolyl, thiazolyl, indolyl, pyrryl, oxazolyl, benzofuryl, benzothienyl, benzthiazolyl, isoxazolyl, pyrazolyl, triazolyl, tetrazolyl, indazolyl, 1,2,4-thiadiazolyl, isothiazolyl, benzothienyl, purinyl, carbazolyl, fluorenonyl, benzimidazolyl, indolinyl, and the like. In some embodiments, the heteroaryl group has from 1 to about 20 carbon atoms, and in further embodiments from about 3 to about 20 carbon atoms. In some embodiments, the heteroaryl group contains 3 to about 14, 4 to about 14, 3 to about 7, or 5 to 6 ring-forming atoms. In some embodiments, the heteroaryl or heteroaromatic group has 1 to about 4, 1 to about 3, or 1 to 2 heteroatoms. In some embodiments, the heteroaryl or heteroaromatic group has 1 heteroatom.

The term “4-, 5- or 6-membered heterocyclic ring containing one or more heteroatoms independently selected from N, O, or S” refers to a mono- or bicyclic-heterocyclic ring which may be saturated or partly saturated and which may optionally contain a carbonyl function and which may be, but is not limited to, azetidinyl, imidazolidinyl, imidazolinyl, morpholinyl, piperazinyl, piperidinyl, piperidonyl, pyrazolidinyl, pyrazolinyl, pyrrolidinyl, pyrrolinyl, 1-methyl-1,4-diazepane, tetrahydropyranyl or thiomorpholinyl. In the case where the heterocyclic ring contains a heteroatom selected from S or N, these atoms may optionally be in an oxidised form such as SO or SO₂.

The term “hydrochloride” includes monohydrochloride, dihydrochloride, trihydrochloride and tetrahydrochloride salts.

A suitable pharmaceutically acceptable salt of the compound of the invention is, for example, an acid-addition salt, for example an inorganic or organic acid. In addition a suitable pharmaceutically acceptable salt of the compounds of the invention is an alkali metal salt, an alkaline earth metal salt or a salt with an organic base that affords a physiologically-acceptable cation.

Some compounds of formula I may have stereogenic centres and/or geometric isomeric centres (E- and Z-isomers), and it is to be understood that the invention encompasses all such optical, diastereoisomers and geometric isomers.

The present invention relates to the use of compounds of formula I as hereinbefore defined as well as to the salts thereof. Salts for use in pharmaceutical compositions will be pharmaceutically acceptable salts, but other salts may be useful in the production of the compounds of formula I.

It is to be understood that the present invention relates to any and all tautomeric forms of the compounds of formula I.

An object of the invention is to provide compounds of formula I for therapeutic use, especially compounds that are useful for the prevention and/or treatment of conditions associated with glycogen synthase kinase-3 (GSK3) in mammals including man. Particularly, compounds of formula I exhibiting a selective affinity for GSK-3.

Another aspect of the invention is wherein a compound of formula I or a pharmaceutically acceptable salt, solvate or in vivo hydrolysable ester thereof, or a pharmaceutical composition or formulation comprising a compound of formula I is administered concurrently, simultaneously, sequentially or separately with another pharmaceutically active compound or compounds selected from the following:

(i) antidepressants such as agomelatine, amitriptyline, amoxapine, bupropion, citalopram, clomipramine, desipramine, doxepin duloxetine, elzasonan, escitalopram, fluvoxamine, fluoxetine, gepirone, imipramine, ipsapirone, maprotiline, nortriptyline, nefazodone, paroxetine, phenelzine, protriptyline, ramelteon, reboxetine, robalzotan, sertraline, sibutramine, thionisoxetine, tranylcypromaine, trazodone, trimipramine, venlafaxine and equivalents and pharmaceutically active isomer(s) and metabolite(s) thereof.
(ii) atypical antipsychotics including for example quetiapine and pharmaceutically active isomer(s) and metabolite(s) thereof.
(iii) antipsychotics including for example amisulpride, aripiprazole, asenapine, benzisoxidil, bifeprunox, carbamazepine, clozapine, chlorpromazine, debenzapine, divalproex, duloxetine, eszopiclone, haloperidol, iloperidone, lamotrigine, loxapine, mesoridazine, olanzapine, paliperidone, perlapine, perphenazine, phenothiazine, phenylbutylpiperidine, pimozide, prochlorperazine, risperidone, sertindole, sulpiride, suproclone, suriclone, thioridazine, trifluoperazine, trimetozine, valproate, valproic acid, zopiclone, zotepine, ziprasidone and equivalents and pharmaceutically active isomer(s) and metabolite(s) thereof.
(iv) anxiolytics including for example alnespirone, azapirones, benzodiazepines, barbiturates such as adinazolam, alprazolam, balezepam, bentazepam, bromazepam, brotizolam, buspirone, clonazepam, clorazepate, chlordiazepoxide, cyprazepam, diazepam, diphenhydramine, estazolam, fenobam, flunitrazepam, flurazepam, fosazepam, lorazepam, lormetazepam, meprobamate, midazolam, nitrazepam, oxazepam, prazepam, quazepam, reclazepam, tracazolate, trepipam, temazepam, triazolam, uldazepam, zolazepam and equivalents and pharmaceutically active isomer(s) and metabolite(s) thereof.
(v) anticonvulsants including for example carbamazepine, valproate, lamotrogine, gabapentin and equivalents and pharmaceutically active isomer(s) and metabolite(s) thereof.
(vi) Alzheimer's therapies including for example donepezil, memantine, tacrine and equivalents and pharmaceutically active isomer(s) and metabolite(s) thereof.
(vii) Parkinson's therapies including for example deprenyl, L-dopa, Requip, Mirapex, MAOB inhibitors such as selegine and rasagiline, comP inhibitors such as Tasmar, A-2 inhibitors, dopamine reuptake inhibitors, NMDA antagonists, Nicotine agonists, Dopamine agonists and inhibitors of neuronal nitric oxide synthase and equivalents and pharmaceutically active isomer(s) and metabolite(s) thereof.
(viii) migraine therapies including for example almotriptan, amantadine, bromocriptine, butalbital, cabergoline, dichloralphenazone, eletriptan, frovatriptan, lisuride, naratriptan, pergolide, pramipexole, rizatriptan, ropinirole, sumatriptan, zolmitriptan, zomitriptan, and equivalents and pharmaceutically active isomer(s) and metabolite(s) thereof.
(ix) stroke therapies including for example abciximab, activase, NXY-059, citicoline, crobenetine, desmoteplase, repinotan, traxoprodil and equivalents and pharmaceutically active isomer(s) and metabolite(s) thereof.
(x) urinary incontinence therapies including for example darafenacin, falvoxate, oxybutynin, propiverine, robalzotan, solifenacin, tolterodine and equivalents and pharmaceutically active isomer(s) and metabolite(s) thereof.
(xi) neuropathic pain therapies including for example gabapentin, lidoderm, pregablin and equivalents and pharmaceutically active isomer(s) and metabolite(s) thereof.
(xii) nociceptive pain therapies such as celecoxib, etoricoxib, lumiracoxib, rofecoxib, valdecoxib, diclofenac, loxoprofen, naproxen, paracetamol and equivalents and pharmaceutically active isomer(s) and metabolite(s) thereof.
(xiii) insomnia therapies including for example agomelatine, allobarbital, alonimid, amobarbital, benzoctamine, butabarbital, capuride, chloral, cloperidone, clorethate, dexclamol, ethchlorvynol, etomidate, glutethimide, halazepam, hydroxyzine, mecloqualone, melatonin, mephobarbital, methaqualone, midaflur, nisobamate, pentobarbital, phenobarbital, propofol, ramelteon, roletamide, triclofos, secobarbital, zaleplon, zolpidem and equivalents and pharmaceutically active isomer(s) and metabolite(s) thereof.
(xiv) mood stabilizers including for example carbamazepine, divalproex, gabapentin, lamotrigine, lithium, olanzapine, quetiapine, valproate, valproic acid, verapamil, and equivalents and pharmaceutically active isomer(s) and metabolite(s) thereof.

Such combination products employ the compounds of this invention within the dosage range described herein and the other pharmaceutically active compound or compounds within approved dosage ranges and/or the dosage described in the publication reference.

### Methods of Preparation

Another aspect of the present invention provides a process for preparing a compound of formula I as a free base or a pharmaceutically acceptable salt thereof. Throughout the following description of such processes it is understood that, where appropriate, suitable protecting groups will be added to, and subsequently removed from, the various reactants and intermediates in a manner that will be readily understood by one skilled in the art of organic synthesis. Conventional procedures for using such protecting groups as well as examples of suitable protecting groups are described, for example, in “Protective Groups in Organic Synthesis”, T. W. Greene, P. G. M. Wuts, Wiley-Interscience, New York, 1999. It will be appreciated that certain of the various ring substituents in the compounds of the present invention may be introduced by standard aromatic substitution reactions or generated by conventional functional group modifications either prior to or immediately following the processes mentioned above, and as such are included in the process aspect of the invention. Such reactions and modifications include, for example, introduction of a substituent by means of an aromatic substitution reaction, reduction of substituents, alkylation of substituents and oxidation of substituents. The reagents and reaction conditions for such procedures are well known in the chemical art. The introduction of an acyl group using, for example, an acyl halide and Lewis acid (such as aluminium trichloride) under Friedel Crafts conditions; the introduction of an alkyl group using an alkyl halide and Lewis acid (such as aluminium trichloride) under Friedel Crafts conditions; and the introduction of a halogeno group. Particular examples of modifications include the reduction of a nitro group to an amino group by for example, catalytic hydrogenation with a nickel catalyst or treatment with iron in the presence of hydrochloric acid with heating; oxidation of alkylthio to alkylsulphinyl or alkylsulphonyl.

### Methods of Preparation of Intermediates

The processes for the preparation of the intermediates, wherein R¹, R², R³, R⁴, R⁵, R⁶, R⁷, R^{b} and R^{c} are, unless specified otherwise, defined as in formula I and Q is halo, comprise of the following:

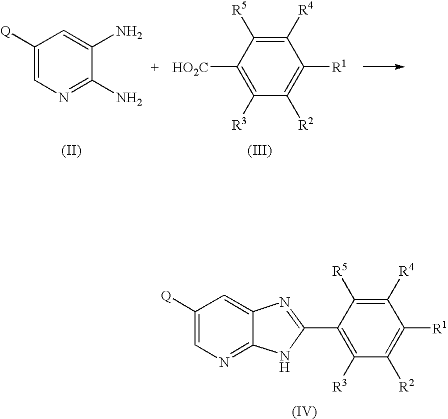

(i) Condensation of diamine II with a carboxylic acid of type III to give an intermediate IV can be performed by
(a) First, reacting II and III in the presence of a suitable catalyst, e.g. o-benzotriazol-1-yl-N,N,N′,N′-tetramethyluroniumhexafluorophosphate or O-(7-azabenzotriazol-1-yl)-N,N,N′,N′-tetramethyluronium hexafluorophosphate, in a solvent such as acetonitrile, dimethyl formamide, or a mixture thereof. A suitable base such as N,N-diisopropylethylamine may be used in the reaction, which can be performed at a temperature in the range of 0° C. to +20° C.
(b) Second, heating the resulting intermediate in a suitable organic acid such as acetic acid, at a temperature in the range of +150° C. to +200° C. using an oil bath or a microwave oven.

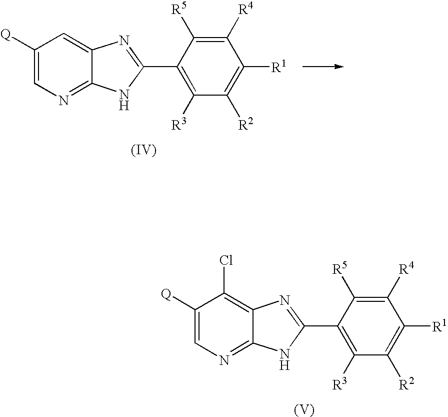

(ii) Conversion of a compound of type IV into a chloride of type V can be achieved by (a) first, reacting IV with an appropriate oxidant, e.g. m-chloroperbenzoic acid, in a suitable solvent, e.g. acetic acid, at a temperature in the range of +20° C. to +30° C.; (b) second, reaction of the formed intermediate with neat phosphorus oxychloride at a temperature in the range of +100° C. to +150° C. using an oil bath or a microwave oven.

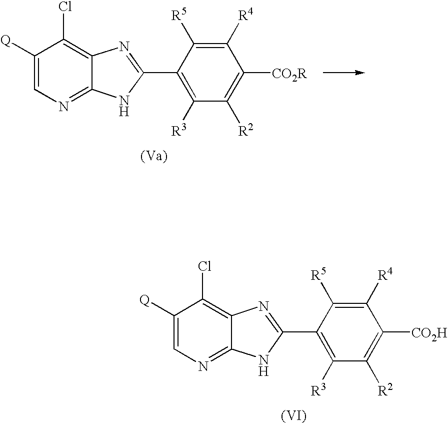

(iii) Hydrolysis of an ester of type Va (V, wherein R¹ is CO₂R and wherein R is methyl) to the corresponding acid VI might be effected by reaction with a suitable base, such as lithium, sodium or potassium hydroxide, or potassium carbonate, in mixtures of water and a suitable co-solvent, e.g. tetrahydrofuran or methanol, at a temperature in the range of +20° C. to +120° C. using an oil bath or a microwave oven.

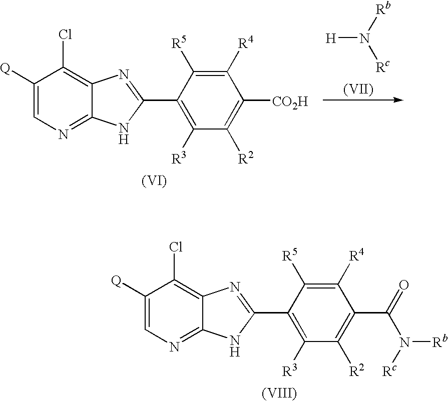

(iv) Formation of an amide of type VIII from the corresponding acid VI and an amine VII can be performed by reacting VI and VII in the presence of a suitable catalyst, e.g. o-benzotriazol-1-yl-N,N,N′,N′-tetramethyluroniumhexafluorophosphate or O-(7-azabenzotriazol-1-yl)-N,N,N′,N′-tetramethyluronium hexafluorophosphate, in a solvent such as acetonitrile, dimethyl formamide, or a mixture thereof. A suitable base such as N,N-diisopropylethylamine may be used in the reaction, which can be performed at a temperature in the range of 0° C. to +20° C. Alternatively, a solution of VI in a solvent such as dimethyl acetamide can be first reacted with 1,1′-carbonylbis(1H-imidazole) at a temperature in the range of +80° C. to +120° C., and then reacted with the amine VII at a temperature in the range of +100° C. to +150° C. using an oil bath or a microwave oven.

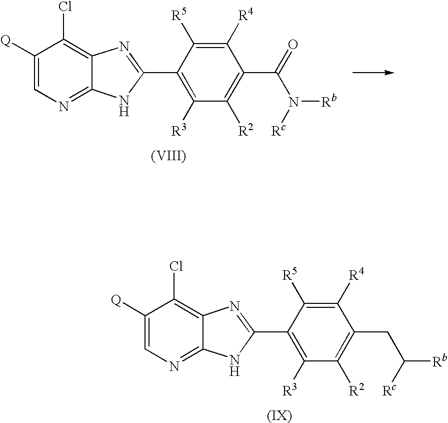

(v) A compound of type VIII can be transformed into a compound of type IX by reaction with a suitable reducing agent, e.g. borane, in a suitable solvent such as tetrahydrofuran, at a temperature in the range of 0° C. to +60° C.

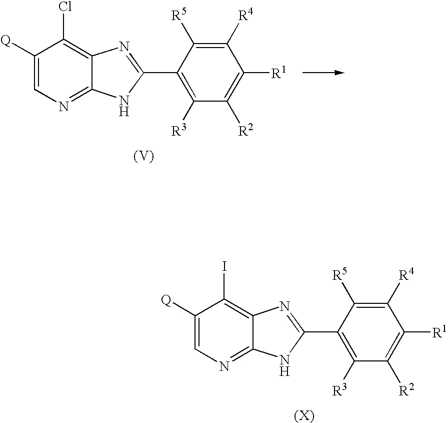

(vi) A compound of type V can be transformed into the corresponding iodide X by (a) first, treatment with HCl in a suitable solvent such as diethyl ether to give the hydrochloride salt, and (b) second, reaction of the salt with NaI in a suitable solvent, e.g. acetonitrile, at a temperature in the range of +150° C. to +175° C. using an oil bath or a microwave oven.

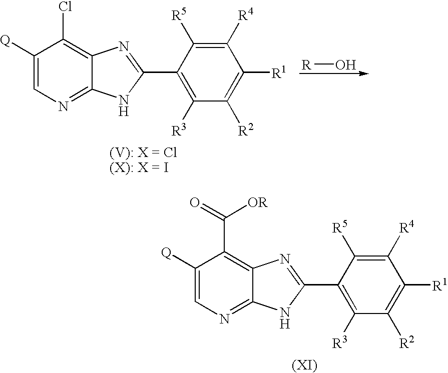

(vii) A compound of type V or X may be transformed into a compound of type XI as described below: (a) in the presence of a suitable catalyst, e.g. PdCl₂(dppf), suitable alcohol (ROH), co-reagents such as 1,8-diazabicyclo[5.4.0]undec-7-ene and imidazole, and molybdenum hexacarbonyl, the reaction can be carried out in a suitable solvent such as THF or alcohol (ROH) by heating to a temperature in the range of +125° C. to +175° C. in a microwave oven;
(b) in the presence of a suitable catalyst, e.g. Pd(OAc)₂/1,3-bis(diphenylphosphino)propane or PdCl₂(BINAP), the reaction is run in an autoclave under a pressure of carbon monoxide of 1-5 bar, in a suitable solvent such as dioxane or alcohol (ROH), and at a temperature in the range of +80° C. to +120° C.

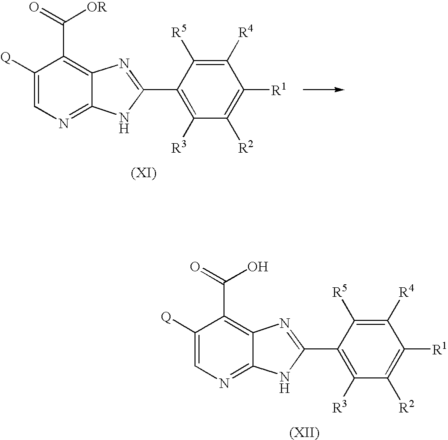

(viii) Hydrolysis of an ester of type XI (wherein R is alkyl e.g. methyl or ethyl) to the corresponding acid XII might be effected by reaction with a suitable base, such as lithium, sodium or potassium hydroxide, or potassium carbonate, in mixtures of water and a suitable co-solvent, e.g. tetrahydrofuran or methanol, at a temperature in the range of +20° C. to +120° C. using an oil bath or a microwave oven.

### Methods of Preparation of End Products

Another objective of the invention are processes for the preparation of a compound of general formula I, wherein R¹, R², R³, R⁴, R⁵, R⁶, R⁷, R^{b} and R^{c} are, unless specified otherwise, defined as in formula I and Q is halo, comprises of:

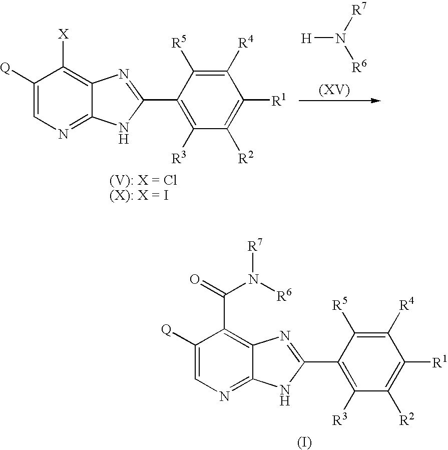

(i) A compound of type V or X may be coupled with an amine XV to give a compound of type I as described below: (a) in the presence of a suitable catalyst, e.g. PdCl₂(dppf), suitable amine co-reagents such as 1,8-diazabicyclo[5.4.0]undec-7-ene and imidazole, and to molybdenum hexacarbonyl, the reaction can be carried out in a suitable solvent such as THF by heating to a temperature in the range of +125° C. to +175° C. in a microwave oven;
(b) in the presence of a suitable catalyst, e.g. Pd(OAc)₂/1,3-bis(diphenylphosphino)propane or PdCl₂(BINAP), the reaction is run in an autoclave under a pressure of carbon monoxide of 1-5 bar, in a suitable solvent such as dioxane, and at a temperature in the range of +80° C. to +120° C.

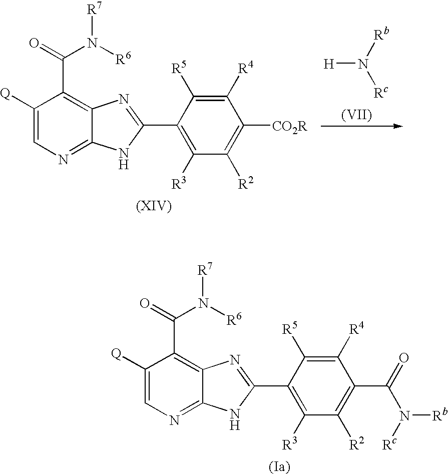

(ii) An ester of type XIV (wherein R is alkyl e.g. methyl or ethyl) may be transformed into a compound of type Ia (I, A=CONR^{b}R^{c}) by (a) first, heating neat with an amine VII at a temperature in the range of +180° C. to +220° C. using an oil bath or a microwave oven, and (b) second, after cooling, adding a suitable catalyst such as o-benzotriazol-1-yl-N,N,N′,N′-tetramethyluroniumhexafluorophosphate or O-(7-azabenzotriazol-1-yl)-N,N,N′,N′-tetramethyluronium hexafluorophosphate, and continuing the reaction at a temperature in the range of 0° C. to +20° C.

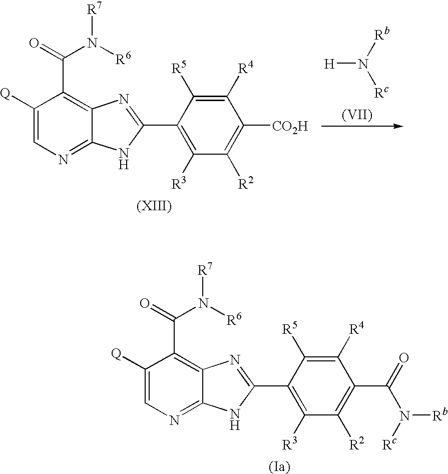

(iii) Coupling of a carboxylic acid of type XIII with an amine of type XIV to give Ia can be performed as described above for the preparation of VIII from VI and VII.

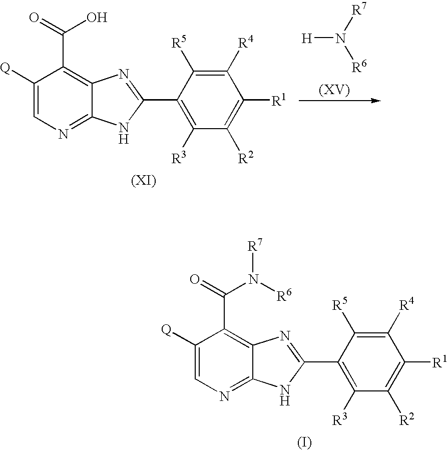

(iv) Coupling of a carboxylic acid of type XI with an amine of type XV to give a compound of type I can be performed as described above for the preparation of VIII from VI and VII.

Consequently, in one aspect of the present invention, there is provided a process for preparing a compound of formula I, wherein—R¹, R², R³, R⁴, R⁵, R⁶, R⁷, R^{b} and R^{c} are, unless specified otherwise, defined as in formula I and Q is halo, comprising of:

(i) Metal-catalyzed carbonylative coupling of a compound of type V or X with an amine XV, using molybdenum hexacarbonyl or carbon monoxide gas, optionally with added amine co-reagents.
(ii) An ester of type XIV may be coupled with an amine VII to give a compound of type Ia (I, A=CONR^{b}R^{c}) by first heating XIV with the neat amine VII, and then adding a suitable catalyst and continuing the reaction.
(iii) Formation of an amide of type Ia can also be performed by reacting a carboxylic acid of type XIII with an amine of type VII, in the presence of a suitable catalyst, optionally with an added amine base. Alternatively, the acid XIII can be first reacted with an activating agent, and then reacted with the amine.
(iv) Formation of an amide of type I can also be performed by reacting a carboxylic acid of type XI with an amine of type XV, in the presence of a suitable catalyst, optionally with an added amine base. Alternatively, the acid XI can be first reacted with an activating agent, and then reacted with the amine.

The hydrochloric salt of a compound of formula I may be obtained from a compound of formula I by treatment with hydrochloric acid at a temperature range between 0° C. and +25° C., in a suitable solvent such as dichloromethane, tetrahydrofuran or dichloromethane/methanol mixture.

### General Methods

All solvents used were analytical grade and commercially available anhydrous solvents were routinely used for reactions. Reactions were typically run under an inert atmosphere of nitrogen or argon.

¹H, ¹⁹F and ¹³C NMR spectra were recorded at 400 MHz for proton, 376 MHz for fluorine-19 and 100 MHz for carbon-13, either on a Varian Unity+400 NMR Spectrometer equipped with a 5 mm BBO probehead with Z-gradients, or a Bruker Avance 400 NMR spectrometer equipped with a 60 μl dual inverse flow probehead with Z-gradients, or a Bruker DPX400 NMR spectrometer equipped with a 4-nucleus probehead equipped with Z-gradients, or a Bruker Avance 600 NMR spectrometer equipped with a 5 mm BBI probehead with Z-gradients. Unless specifically noted in the examples, spectra were recorded at 400 MHz for proton, 376 MHz for fluorine-19 and 100 MHz for carbon-13. The following reference signals were used: the middle line of DMSO-d₆ δ 2.50 (1H), δ 39.51 (¹³C); the middle line of CD₃OD δ 3.31 (1H) or δ 49.15 (¹³C), CDCl₃ δ 7.26 (1H) and the middle line of CDCl₃ δ 77.16 (¹³C) (unless otherwise indicated).

Mass spectra were recorded on a Waters LCMS consisting of an Alliance 2795 (LC), Waters PDA 2996 and a ZQ single quadrupole mass spectrometer. The mass spectrometer was equipped with an electrospray ion source (ESI) operated in a positive or negative ion mode. The capillary voltage was 3 kV and cone voltage was 30 V. The mass spectrometer was scanned between m/z 100-700 with a scan time of 0.3 s. Separations were performed on either Waters X-Terra MS C8 (3.5 μm, 50 or 100 mm×2.1 mm i.d.) or an ACE 3 AQ (100 mm×2.1 mm i.d.) obtained from ScantecLab. Flow rates were regulated to 1.0 or 0.3 mL/min, respectively. The column temperature was set to 40° C. A linear gradient was applied using a neutral or acidic mobile phase system, starting at 100% A (A:95:5 0.1M NH₄OAc:MeCN or 95:5 8 mM HCOOH:MeCN) ending at 100% B (MeCN).

Alternatively, mass spectra were recorded on a Waters LC-MS system (Sample Manager 2777C, 1525μ binary pump, 1500 Column Oven, ZQ, PDA2996 and ELS detector, Sedex 85). Separation was performed using a Zorbax column (C8, 3.0×50 mm, 3 μm). A four minutes linear gradient was used starting at 100% A (A: 95:5 10 mM NH₄OAc:MeOH) and ending at 100% B (MeOH). The ZQ was equipped with a combined APPI/APCI ion source and scanned in the positive mode between m/z 120-800 with a scan time of 0.3 s. The APPI repeller and the APCI corona were set to 0.86 kV and 0.80 μA, respectively. In addition, the desolvation temperature (300° C.), desolvation gas (400 L/Hr) and cone gas (5 L/Hr) were constant for both APCI and APPI mode.

Alternatively, mass spectra were recorded on a Waters LCMS consisting of an Alliance 2690 Separations Module, Waters 2487 Dual 1 Absorbance Detector (220 and 254 nm) and a Waters ZQ single quadrupole mass spectrometer. The mass spectrometer was equipped with an electrospray ion source (ESI) operated in a positive or negative ion mode. The capillary voltage was 3 kV and cone voltage was 30 V. The mass spectrometer was scanned between m/z 97-800 with a scan time of 0.3 or 0.8 s. Separations were performed on a Chromolith Performance RP-18e (100×4.6 mm). A linear gradient was applied starting at 95% A (A: 0.1% HCOOH (aq.)) ending at 100% B (MeCN) in 5 minutes. Flow rate: 2.0 mL/min.

Microwave heating was performed in a Creator or Smith Synthesizer Single-mode microwave cavity producing continuous irradiation at 2450 MHz. HPLC analyses were performed on an Agilent HP1000 system consisting of G1379A Micro Vacuum Degasser, G1312A Binary Pump, G1367A Well plate auto-sampler, G1316A Thermostatted Column Compartment and G1315B Diode Array Detector. Column: X-Terra MS, Waters, 3.0×100 mm, 3.5 μm. The column temperature was set to 40° C. and the flow rate to 1.0 ml/min. The Diode Array Detector was scanned from 210-300 nm, step and peak width were set to 2 nm and 0.05 min, respectively. A linear gradient was applied, starting at 100% A (95:5 10 mM NH₄OAc:MeCN) and ending at 100% B (B: acetonitrile), in 4 min.

A typical workup procedure after a reaction consisted of extraction of the product with a solvent such as ethyl acetate, washing with water followed by drying of the organic phase over MgSO₄ or Na₂SO₄, filtration and concentration of the solution in vacuo.

Thin layer chromatography (TLC) was performed on Merck TLC-plates (Silica gel 60 F₂₅₄) and UV visualized the spots. Flash chromatography was preformed on a Combi Flash® Companion™ using RediSep™ normal-phase flash columns. Typical solvents used for flash chromatography were mixtures of chloroform/methanol, dichloromethane/methanol, heptane/ethyl acetate, chloroform/methanol/ammonia (aq.) and dichlorormethane/methanol/ammonia (aq.). SCX ion exchange columns were performed on Isolute® columns. Chromatography through ion exchange columns were typically performed in solvents such a methanol. or 10% ammonia in methanol.

Preparative chromatography was run on a Waters autopurification HPLC with a diode array detector. Column: XTerra MS C8, 19×300 mm, 10 μm. Narrow gradients with MeCN/(95:5 0.1M NH₄OAc:MeCN) were used at a flow rate of 20 ml/min. Alternatively, purification was achieved on a semi preparative Shimadzu LC-8A HPLC with a Shimadzu SPD-10A UV-vis.-detector equipped with a Waters Symmetry® column (C18, 5 μm, 100 mm×19 mm). Narrow gradients with MeCN/0.1% trifluoroacetic acid in MilliQ Water were used at a flow rate of 10 ml/min.

The formation of hydrochloride salts of the final products were typically performed by dissolution in solvents or solvent mixtures such as diethyl ether, tetrahydrofuran, dichloromethane/methanol, followed by addition of 1M HCl in diethyl ether.

The following abbreviations have been used:
AIBN 2,2′-azobis(2-methylpropionitrile);
aq. aqueous;
CH₂Cl₂ dimethyl chloride;
BINAP 2,2′-bis(diphenylphosphino)-1,1′binaphtyl;
DBU 1,8-Diazabicyclo[5.4.0]undec-7-ene;
DIPEA diisopropylethylamine;
DMF N—N-dimethylformamide;
ether diethyl ether;
Et₂O diethyl ether;
EtOAc ethyl acetate;
EtOH ethanol;
HBTU o-benzotriazol-1-yl-N,N,N′,N′-tetramethyluroniumhexafluorophosphate;
HCl hydrochloride;
HOAc acetic acid;
(i-Pr)₂NEt N—N-diisopropylethylamine;
m-CPBA 3-chloroperoxybenzoic acid;
MeCN acetonitrile;
MeOH methanol;
MgSO₄ magnesium sulphate;
Mo(CO)₆ molybdenum hexacarbonyl
NaHCO₃ sodium bicarbonate;
NaI sodium iodide;
Na₂SO₄ sodium sulphate;
Na₂S₂O₃ sodium thiosulphate
NH₄OAc ammonium acetate;
Pd(OAc)₂ palladium diacetate;
PdCl₂(dppf)*DCM (1,1′-bis(diphenylphosphino)ferrocen)palladium(II) chloride dichlorometane adduct;
Pd(dppf)Cl₂ 1,1′-bis(diphenylphosphino)ferrocene palladium(II) chloride;
PdCl₂(BINAP) 2,2′-bis(diphenylphospine)-1,1′-binaphtyl palladium (II) dichloride
POCl₃ phosphoroxidchloride
r.t. room temperature;
THF tetrahydrofuran.

Compounds have been named either using ACD/Name, version 8.08, software from Advanced Chemistry Development, Inc. (ACD/Labs), Toronto ON, Canada, www.acdlabs.com, 2004 or named according to the IUPAC convention.

### WORKING EXAMPLES

Below follows a number of non-limiting examples of the compounds of the present invention.

### Example 1

### Methyl 4-(6-bromo-3H-imidazo[4,5-b]pyridin-2-yl)benzoate

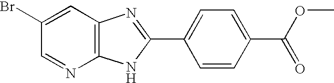

DIPEA (16.6 mL, 95.7 mmol) was added to a suspension of 5-bromo pyridine-2,3-diamine (6.0 g, 31.9 mmol), terepthalic acid monomethyl ester (6.89 g, 38.3 mmol) and HBTU (14.5 g, 38.3 mmol) in MeCN (100 mL) and the reaction mixture was stirred at r.t. for 1 h. A precipitate that formed was collected and washed with MeCN. The solid was distributed into microwave vials with HOAc (4 mL) and heated to +200° C. for 5 minutes. The product precipitated at r.t. and was filtered, washed with HOAc and MeCN and dried to afford 8.58 g (81% yield) of the title compound.

¹H NMR (CDCl₃) δ ppm); 8.15 (d, J=1.52 Hz, 1H), 8.07-8.09 (m, 2H), 7.97 (d, J=8.84 Hz, 2H), 7.59 (d, J=1.52 Hz, 1H), 3.75 (s, 3H); MS (APPI) m/z (M+1) 332, 334.

### Example 2

### Methyl 4-(6-chloro-3H-imidazo[4,5-b]pyridin-2-yl)benzoate

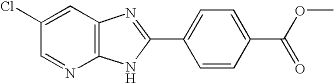

DIPEA (21.9 mL, 126 mmol) was added to a suspension of 5-chloro pyridine-2,3-diamine (6.0 g, 42.0 mmol), terepthalic acid monomethyl ester (9.06 g 50.3 mmol) and HBTU (19.1 g 50.3 mmol) in MeCN (100 mL) and the reaction mixture was stirred at r.t. for 1 h. A precipitatethat formed was collected and washed with MeCN. The solid was distributed intomicrowave vials with HOAc (4 mL) and heated to +200° C. for 10 minutes. The productprecipitated at r.t. and was filtered, washed with HOAc and MeCN and dried to to afford 10.3 g (85% yield) of the title compound.

¹H NMR (CDCl₃) δ ppm); 7.92-7.98 (m, 3H), 7.84 (d, J=8.84 Hz, 2H), 7.38 (d, J=1.77 Hz, 1H), 3.59-3.63 (m, 3H); MS (APPI) m/z (M+1) 288, 290.

### Example 3

### Methyl 4-(6-bromo-7-chloro-3H-imidazo[4,5-b]pyridin-2-yl)benzoate

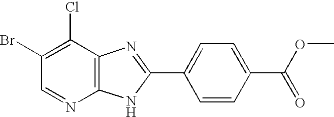

Methyl 4-(6-bromo-3H-imidazo[4,5-b]pyridin-2-yl)benzoate (6.7 g, 20.2 mmol), which was obtained from Example 1 and m-CPBA (70%, 17.75 g, 60.3 mmol) in HOAc was stirred at r.t. for 18 h. An additional 2 equvalents of m-CPBA (70%, 9.06 g, 40.6 mmol) was added to the reaction mixture, and stirring was continued for 6 h. The solvent was evaporated in vacuo and the residue was crystallized from EtOH. The solid was mixed with POCl₃ and heated in a microwave reactor at +120° C. for 5 minutes. After cooling to r.t., the mixture was poured into ice/water mixture and the precipitate that formed was collected, washed with water and dried, affording the title compound in 6.1 g (83%) yield. ¹H NMR (400 MHz, DMSO-d₆) δ ppm; 8.59 (s, 1H), 8.40 (d, J=8.53 Hz, 2H), 8.15 (d, J=8.53 Hz, 2H), 3.91 (s, 3H); MS (APPI) m/z (M+1) 368.

### Example 4

### Methyl 4-(6,7-dichloro-3H-imidazo[4,5-b]pyridin-2-yl)benzoate

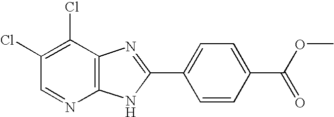

Methyl 4-(6-chloro-3H-imidazo[4,5-b]pyridin-2-yl)benzoate (8.3 g, 28.8 mmol), which was obtained from Example 2 and m-CPBA (70%, 19.4 g, 86.5 mmol) in HOAc was stirred at r.t. for 18 h. An additional 2 equvalents of m-CPBA (70%, 9.06 g, 40.6 mmol) was added to the reaction mixture, and stirring was continued for 6 h. The solvent was evaporated in vacuo and the residue was crystallized from EtOH. The solid was mixed with POCl₃ and heated in a microwave reactor at +120° C. for 5 minutes. After cooling to r.t., the mixture was poured into ice/water mixture and the precipitate that formed was collected, washed with water and dried, affording the title compound in 9.3 g (81%) yield. ¹H NMR (400 MHz, DMSO-d₆) δ ppm; 8.53 (s, 1H), 8.41 (d, J=8.53 Hz, 2H), 8.15 (d, J=8.53 Hz, 2H), 3.91 (s, 3H); MS (APPI) m/z (M+1) 322, 324.

### Example 5

### Methyl 4-(6-bromo-7-iodo-3H-imidazo[4,5-b]pyridin-2-yl)benzoate

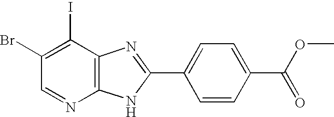

Methyl 4-(6-bromo-7-chloro-3H-imidazo[4,5-b]pyridin-2-yl)benzoate (2.0 g, 5.46 mmol), obtained from Example 3 was dissolved in CH₂Cl₂/MeOH (9:1, 20 mL), and HCl (1M in Et₂O, 2 mL) was added followed by addition of Et₂O until a precipitate formed. The solid was collected by filtration and dried. The hydrochloride was mixed with sodium iodide (16.4 g, 109 mmol) and MeCN 10 (mL) and heated in a microwave reactor at +160° C. for 20 minutes. The mixture was added to Na₂S₂O₃ (10%, aq.). The precipitate was filtered and washed with water and dried in vacuo to afford 1.95 g (78%) yield of the title compound. The mixture was used in the next step without further purification.

¹H NMR (400 MHz, DMSO-d₆) δ ppm 8.36-8.45 (m, 3H) 8.13 (d, 2H) 3.91 (s, 3H); MS (ESI) m/z (M+1) 458, 460.

### Example 6

### Methyl 4-(6-chloro-7-iodo-3H-imidazo[4,5-b]pyridin-2-yl)benzoate

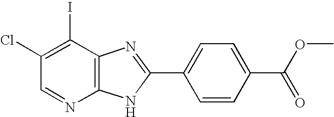

Methyl 4-(6,7-dichloro-3H-imidazo[4,5-b]pyridin-2-yl)benzoate (2.0 g, 6.2 mmol), obtained from Example 4 was dissolved in CH₂Cl₂/MeOH (9:1, 20 mL), and HCl (1M in Et₂O, 2 mL) was added followed by addition of Et₂O until a precipitate formed. The solid was collected by filtration and dried. The hydrochloride was mixed with sodium iodide (18.6 g, 124 mmol) and MeCN 10 (mL) and heated in a microwave reactor at +160° C. for 20 minutes. The mixture was added to Na₂S₂O₃ (10%, aq.). The precipitate was filtered and to washed with water and dried in vacuo to afford 1.9 g (76%) yield of the title compound. The mixture was used in the next step without further purification.

¹H NMR (400 MHz, DMSO-d₆) δ ppm; 8.40 (d, J=8.53 Hz, 1H), 8.34 (s, 1H), 8.13 (d, J=8.28 Hz, 2H), 3.90 (s, 3H); MS (APPI) m/z (M+1) 414, 416

### Example 7

### 4-(6-Bromo-7-iodo-3H-imidazo[4,5-b]pyridin-2-yl)benzoic acid

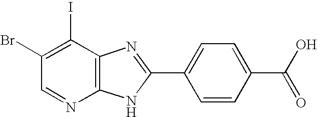

Methyl 4-(6-bromo-7-iodo-3H-imidazo[4,5-b]pyridin-2-yl)benzoate (as described in Example 5) (4.5 g, 9.8 mmol) and lithium hydroxide hydrate (4.2 g, 100 mmol) was dissolved in THF:water 9:1 (45 ml). The mixture was divided into three vials and was heated at 100° C. by microwave irradiation for 10 minutes. The reaction mixture was made acidic by the addition of hydrochloric acid (2M). The solid formed was collected by filtration and washed with water and dried under vacuum to give the title compound (3.6 g, 82%) which was used without further purification. MS (ESI) m/z (M−1) 442; 444.

### Example 8

### 4-(6-Chloro-7-iodo-3H-imidazo[4,5-b]pyridin-2-yl)benzoic acid

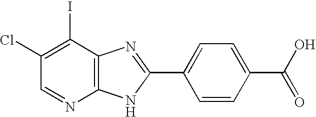

Methyl 4-(6-Chloro-7-iodo-3H-imidazo[4,5-b]pyridin-2-yl)benzoate (as described in Example 6) (1.13 g, 2.74 mmol) and lithiumhydroxide hydrate (0.23 g, 5.47 mmol) was dissolved in THF:water 9:1 (45 ml). The mixture was heated at 100° C. by microwave irradiation for 10 minutes. The reaction mixture was made acidic by the addition of hydrochloric acid (2M). The solid formed was collected by filtration and washed with water and dried under vacuum to give the title compound (0.95 g, 86%) which was used without further purification.

MS (APPI) m/z (M+1) 400, 402

### Example 9

### 6-Bromo-7-iodo-2-[4-(morpholin-4-ylcarbonyl)phenyl]-3H-imidazo[4,5-b]pyridine

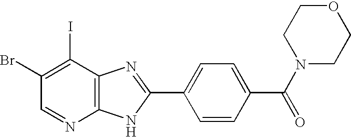

4-(6-Bromo-7-iodo-3H-imidazo[4,5-b]pyridin-2-yl)benzoic acid (as described in Example 7) (1.0 g, 2.25 mmol) and TSTU (0.85 g, 2.81 mmol) were dissolved in DMF (12 ml). Triethylamine (0.94 ml, 6.8 mmol) was added and the mixture was stirred at ambient temperature for 45 minutes. Morpholine (0.39 ml, 4.5 mmol) was added and the mixture was stirred at ambient temperature for 5 h. Aqueous sodium bicarbonate was added and the mixture was extracted with dichloromethane (×3). The organic phase was dried (Na₂SO₄) and concentrated. The crude was purified by column chromatography on silica eluting with gradients of dichloromethane and dichloromethane: methanol; 7N ammonia in methanol 90:10:1 to give the title compound (0.51 g, 44%). A sample for NMR-analysis was purified by preparative HPLC.

¹H NMR (400 MHz, CHLOROFORM-d) δ ppm 8.43 (s, 1H) 8.09 (d, 2H) 7.46 (d, 2H) 3.92 (br. s., 2H) 3.47 (br. s., 2H) 2.57 (br. s., 2H) 2.33-2.46 (m, 5H); MS (ESI) m/z (M+1) 524, 526.

### Example 10

### 6-Chloro-7-iodo-2-[4-(morpholin-4-ylcarbonyl)phenyl]-3H-imidazo[4,5-b]pyridine

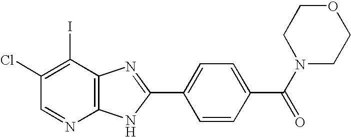

4-(6-Chloro-7-iodo-3H-imidazo[4,5-b]pyridin-2-yl)benzoic acid (as described in Example 8) (0.29 g, 0.73 mmol) and TSTU (0.27 g, 0.91 mmol) were mixed in DMF (3 ml). Triethylamine (0.31 ml, 2.19 mmol) was added and the mixture was stirred at ambient temperature for 1 h. Morpholine (0.13 ml, 1.5 mmol) was added and the mixture was stirred at ambient temperature over night. Aqueous sodium bicarbonate was added and the mixture was extracted with dichloromethane (×2) and ethyl acetate (×1). The combined organic phases were evaporated and the residue was dissolved in DMSO and purified by preparative HPLC. The fractions containing product were pooled and concentrated. Aqueous sodium bicarbonate was added and the mixture was extracted with dichloromethane (×4). The combined organic phases were dried (Na₂SO₄) and concentrated to give the title compound as a solid (0.065 g, 19%).

¹H NMR (400 MHz, CHLOROFORM-d) δ ppm 8.35 (s, 1H) 8.15 (d, 2H) 7.53 (d, 2H) 3.40-3.96 (m, 8H); MS (ESI) m/z (M+1) 469; 471.

### Example 11

### 6-Bromo-7-iodo-2-[4-(morpholin-4-ylmethyl)phenyl]-3H-imidazo[4,5-b]pyridine

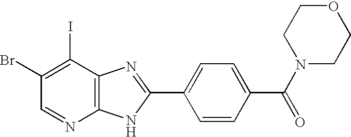

6-Bromo-7-iodo-2-[4-(morpholin-4-ylcarbonyl)phenyl]-3H-imidazo[4,5-b]pyridine (as described in Example 9) (0.48 g, 0.94 mmol) was dissolved in THF (10 ml). Borane (1M in THF, 5 ml) was added. The reaction was stirred at ambient temperature under argon atmosphere for 1.5 h. Methanol (10 ml) was added and the mixture was stirred at ambient temperature for 16 h. The solvent was evaporated and the residue was purified by preparative HPLC. The fractions containing the product were pooled, aqueous sodium bicarbonate was added and the solution was extracted with dichloromethane (×3). The organic phase was dried (Na₂SO₄) and the solvent was evaporated to give the title compound as a solid (45 mg, 10%).

¹H NMR ¹H NMR (400 MHz, DMSO-d₆) δ ppm 14.00 (s, 1H) 8.41 (s, 1H) 8.20 (d, 2H) 7.52 (d, 2H) 3.57-3.62 (m, 4H) 3.55 (s, 2H) 2.39 (br. s., 4H);

MS (ESI) m/z (M+1) 499; 501.

### Example 12

### 6-Chloro-7-iodo-2-[4-(morpholin-4-ylmethyl)phenyl]-3H-imidazo[4,5-b]pyridine

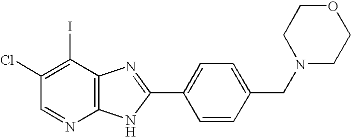

6-Chloro-7-iodo-2-[4-(morpholin-4-ylcarbonyl)phenyl]-3H-imidazo[4,5-b]pyridine (as described in Example 10) (65 mg, 0.14 mmol) was dissolved in THF (4 ml). The mixture was cooled to 0° C. Borane 1 M in THF (1.4 ml, 1.4 mmol) was added. The cooling bath was removed and the mixture was stirred at ambient temperature for 1 h. Methanol (5 ml) was added and the mixture was stirred for 16 h. The solvents were evaporated and the crude (61 mg) was used without further purification.

MS (ESI) m/z (M+1) 455; 457.

### Example 13

### 6-Bromo-S-(3-methoxypropyl)-2-[4-(morpholin-4-ylmethyl)phenyl]-3H-imidazo[4,5-b]pyridine-7-carboxamide

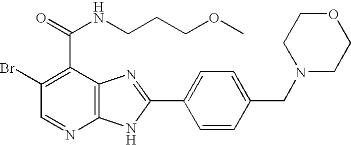

6-Bromo-7-iodo-2-[4-(morpholin-4-ylmethyl)phenyl]-3H-imidazo[4,5-b]pyridine (as described in Example 11) (45 mg, 0.090 mmol) was suspended in dioxane (5 ml) in an autoclave reactor. 3-Methoxypropane-1-amine (0.15 ml), 1,3-bis(diphenylphosphino)propane (4 mg, 0.009 mmol) and Pd(OAc)₂ (1 mg, 0.004 mmol) were added. The vessel was purged with nitrogen followed by carbon monoxide (g). The vessel was pressurized to 5 bars with carbon monoxide (g) and heated to 100° C. for 1 h. The mixture was diluted with dichloromethane and filtered through a plug of diatomeous earth. The mixture was concentrated and the residue was dissolved in DMSO and purified by preparative HPLC. The fractions containing the product were pooled, aqueous sodium bicarbonate was added and the solution was extracted with dichloromethane (×3) and ethyl acetate (×1). The combined organic phases were dried (Na₂SO₄) and the solvent was evaporated. The residue was dissolved in dichloromethane and hydrochloric acid (1M in ether, 0.1 ml) was added. The solvent was evaporated and the hydrochloride salt of the title compound was obtained as a solid (9 mg, 19%).

¹H NMR (400 MHz, DMSO-d₆) δ ppm 10.78 (s, 1H) 8.74 (d, 1H) 8.51 (s, 1H) 8.33 (d, 2H) 7.79 (d, 2H) 4.43 (d, 2H) 4.18-4.29 (m, 1H) 3.96 (d, 2H) 3.74 (t, 2H) 3.24-3.36 (m, 6H) 3.08-3.21 (m, 2H) 1.21 (d, 2H); MS (ESI) m/z (M+1) 488; 490.

### Example 14

### 6-Chloro-N-(3-methoxypropyl)-2-[4-(morpholin-4-ylmethyl)phenyl]-3H-imidazo[4,5-b]pyridine-7-carboxamide

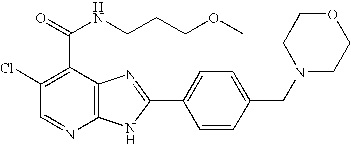

6-Chloro-7-iodo-2-[4-(morpholin-4-ylmethyl)phenyl]-3H-imidazo[4,5-b]pyridine (as described in Example 12) (61 mg, 0.13 mmol) was suspended in dioxane (3 ml) in an autoclave reactor. 3-Methoxypropane-1-amine (0.25 ml), 1,3-bis(diphenylphosphino)propane (5 mg, 0.013 mmol) and Pd(OAc)₂ (1.5 mg, 0.007 mmol) were added. The vessel was purged with nitrogen followed by carbon monoxide (g). The vessel was pressurized to 5 bars with carbon monoxide (g) and heated to 100° C. for 1 h. The mixture was diluted with dichloromethane and filtered through a plug of diatomeous earth. The mixture was concentrated and the residue was dissolved in DMSO and purified by preparative HPLC. The fractions containing the product were pooled, aqueous sodium bicarbonate was added and the solution was extracted with dichloromethane (×4). The organic phase was dried (Na₂SO₄) and the solvent was evaporated. The residue was dissolved in dichloromethane and hydrochloric acid (1M in ether, 0.3 ml) was added. The solvent was evaporated and the hydrochloride salt of the title compound was obtained as a solid (10 mg, 19%).

¹H NMR (400 MHz, DMSO-d₆) δ ppm 10.70 (s, 1H) 8.78 (t, 1H) 8.44 (s, 1H) 8.34 (d, 2H) 7.78 (d, 2H) 4.44 (d, 2H) 3.96 (d, 2H) 3.73 (t, 2H) 3.27 (s, 3H) 3.08-3.20 (m, 2H) 1.76-1.85 (m, 2H) (Peaks obscured by water peak)

MS (APPI) m/z (M+1) 444; 446.

### Example 15

### Methyl 4-(6-fluoro-3H-imidazo[4,5-b]pyridin-2-yl)benzoate

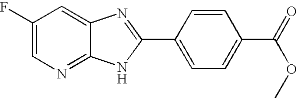

Triethylamine (2.412 mL, 17.31 mmol) was added to a suspension of 5-fluoropyridine-2,3-diamine (2.2 g, 17.31 mmol), 4-(methoxycarbonyl)benzoic acid (3.12 g, 17.31 mmol) and O-benzotriazol-1-yl-tetramethyluronium hexafluorophosphate (6.56 g, 17.31 mmol) in acetonitrile (15 mL) and the reaction mixture was stirred at r.t. for 1 h. The precipitate that formed was collected and washed with MeCN. The solid was distributed into microwave vials with HOAc (4 mL) and heated to 200° C. for 5 minutes. The product precipitated at r.t. and was filtered, washed with HOAc and MeCN and dried to afford methyl 4-(6-fluoro-3H-imidazo[4,5-b]pyridin-2-yl)benzoate (3.70 g, 79%).

MS ESI m/z 272 M+

### Example 16

### Methyl 4-(7-chloro-6-fluoro-3H-imidazo[4,5-b]pyridin-2-yl)benzoate

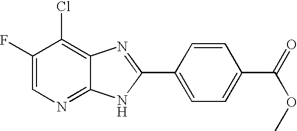

Methyl 4-(6-fluoro-3H-imidazo[4,5-b]pyridin-2-yl)benzoate (2.7 g, 9.95 mmol) and 3-chloroperoxybenzoic acid (7.36 g, 29.86 mmol) were mixed with acetic acid (100 mL) and stirred at R.T for 18 h. The solvent was evaporated. EtOH was added and the product mixture was allowed to stand at R.T o.n. The precipitate was filtered and dried. The intermediate (1.34 g, 4.66 mmol) was suspended in POCl₃ (30 ml). The mixture was heated at 90° for 10 min in a microwave reactor. The mixture was poured onto ice and NaHCO₃ (aq). The solid was isolated by filtration and washed with water. The solid was dried under vacuum at 50° to give a solid (1.2 g, 84%) that was used without further purification. A to sample was purified by preparative HPLC for NMR analysis.

¹H NMR (400 MHz, DMSO-d₆) δ ppm 8.52 (d, 1H) 8.39 (d, 2H) 8.33 (d, 1H) 8.14 (d, 2H) 3.90 (s, 3H)

MS (ESI) m/z 306; 308 (M+1).

### Example 17

### Methyl 4-(6-fluoro-7-iodo-3H-imidazo[4,5-b]pyridin-2-yl)benzoate

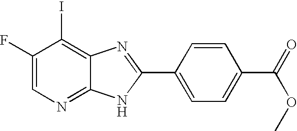

Methyl 4-(7-chloro-6-fluoro-3H-imidazo[4,5-b]pyridin-2-yl)benzoate (1.20 g, 3.93 mmol) was suspended in THF. Hydrochloric acid (1 M in diethyl ether, 4 ml) was added and the solvents were evaporated. Sodium iodide (8.83 g, 58.9 mmol) and acetonitrile (40 ml) was added and the mixture was heated to 160° for 30 min in a microwave reactor. The mixture was poured onto NaHCO₃ (aq) containing Na₂S₂O₃. The solid was collected by filtration and washed with water. The solid was dried under vacuum to give a solid (0.96 g, 62%) that was used without further purification. A sample was purified by preparative HPLC for NMR analysis.

¹H NMR (400 MHz, DMSO-d₆) δ ppm 8.38 (d, 2H) 8.28 (s, 1H) 8.14 (d, 2H) 3.90 (s, 3H)

MS (ESI) m/z 398 (M+1)

### Example 18

### 4-(6-Fluoro-7-iodo-3H-imidazo[4,5-b]pyridin-2-yl)benzoic acid

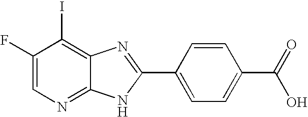

Methyl 4-(6-fluoro-7-iodo-3H-imidazo[4,5-b]pyridin-2-yl)benzoate (0.84 g, 2.12 mmol) and lithium hydroxide monohydrate (0.89 g, 21 mmol) were mixed in THF (18 mL) and to water (2 mL). The mixture was heated to 100° C. for 15 min in a microwave reactor. The mixture was concentrated. Hyrochloric acid (aq, 2M) was added until acidic pH. The solid was isolated by filtration and was washed with water and dried under vacuum to give 0.759 g (94%) which was used without further purification.

MS (ESI) m/z 384 (M+1), m/z 382 (M−1).

### Example 19

### 6-Fluoro-7-iodo-2-[4-(morpholin-4-ylcarbonyl)phenyl]-3H-imidazo[4,5-b]pyridine

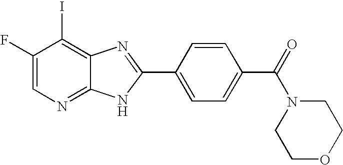

4-(6-Fluoro-7-iodo-3H-imidazo[4,5-b]pyridin-2-yl)benzoic acid (0.730 g, 1.91 mmol) was dissolved in DMF (12 mL). O—(N-Succinimidyl)-N,N,N′,N′-tetramethyl-uronium tetrafluoroborate (0.688 g, 2.29 mmol) and triethylamine (0.80 mL, 5.7 mmol) were added. The mixture was stirred at ambient temperature for 30 minutes. Morpholine (0.25 mL, 2.86 mmol) was added and the mixture was stirred at ambient temperature for 16 h. The mixture was diluted with brine and NaHCO₃ (aq) and was extracted with dichloromethane (×4). The combined organic phases were washed with brine and dried over Na₂SO₄. The solvents were evaporated and the residue was purified by preparative HPLC. The fractions containing product were pooled. NaHCO₃ (aq) was added and the mixture was extracted with dichloromethane (×4). The organic phase was dried (Na₂SO₄) and evaporated to give a solid (0.180 g, 21%).

MS (ESI) m/z 453 (M+1). ¹H NMR (400 MHz, DMSO-d₆) δ ppm 14.08 (s, 1H) 8.32 (d, 2H) 8.24-8.28 (m, 1H) 7.62 (d, 2H) 3.52-3.73 (m, 8H)

### Example 20

### 6-Fluoro-7-iodo-2-[4-(morpholin-4-ylmethyl)phenyl]-3H-imidazo[4,5-b]pyridine

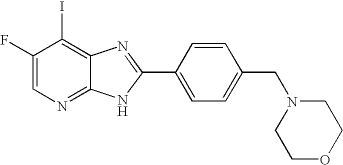

6-Fluoro-7-iodo-2-[4-(morpholin-4-ylcarbonyl)phenyl]-3H-imidazo[4,5-b]pyridine (50 mg, 0.11 mmol) was suspended in THF (2 mL). The mixture was cooled to 0° C. and borane tetrahydrofuran complex (1M in THF, 1.1 mL, 1.1 mmol) was added dropwise. The mixture was stirred at 0° C. for 40 min. The cooling bath was removed and the mixture was stirred at ambient temperature for 1 h. The mixture was cooled to 0° C. and methanol (2 ml) was added. The cooling bath was removed and the mixture was stirred at ambient temperature over night. The solvents were evaporated. Methanol (10 ml) was added and evaporated. The residue (dry film) was used without further purification. ¹H NMR (400 MHz, DMSO-d₆) δ ppm 14.08 (s, 1H) 8.32 (d, 2H) 8.24-8.28 (m, 1H) 7.62 (d, 2H) 3.52-3.73 (m, 8H)

MS (ESI) m/z 439 (M+1).

### Example 21

### 6-Fluoro-N-(3-methoxypropyl)-2-[4-(morpholin-4-ylmethyl)phenyl]-3H-imidazo[4,5-b]pyridine-7-carboxamide hydrochloride

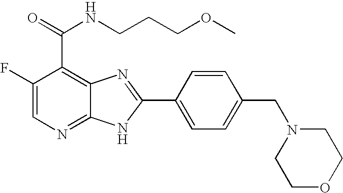

6-Fluoro-7-iodo-2-[4-(morpholin-4-ylmethyl)phenyl]-3H-imidazo[4,5-b]pyridine (48 mg, 0.11 mmol) was dissolved in DMA (0.5 mL) and dioxane (4 mL). 3-Methoxypropylamine (0.1 mL), Pd(OAc)₂ (8 mg, 0.04 mmol) and 1,3-bis(diphenylphosphino)propane (27.1 mg, 0.07 mmol) were added. The vessel was evacuated and pressurized to 5 bar with carbon monoxide. The mixture was heated to 100° C. for 1.5 h. The mixture was filtered through diatomeous earth and the solvent was evaporated. The residue was purified by preparative HPLC. The fractions containing product were pooled. The acetonitrile was removed by evaporation. Hydrochloric acid (1M, aq) was added to acidic pH. The mixture was washed with dichloromethane. NaHCO₃ (aq) was added to neutral pH. The mixture was extracted with dichloromethane (3×10 mL). The combined organic phases were dried (Na₂SO₄) and concentrated. The residue was redissolved in dichloromethane (2 ml). Hydrochloric acid (1M in ether, 0.05 ml) was added. The solvent was evaporated and the hydrochloride salt of the title compound was obtained as a a solid (2.5 mg, 5%).

¹H NMR (400 MHz, DMSO-d₆) δ ppm 8.31-8.41 (m, 1H) 7.76-7.84 (m, 1H) 7.65-7.77 (m, 3H) 7.43-7.56 (m, 2H) 4.13 (dd, 2H) 1.21-1.39 (m, 8H) 0.83-0.90 (m, 6H) MS (ESI) m/z 428 (M+1).

### Pharmaceutical Compositions

According to one aspect of the present invention there is provided a pharmaceutical composition comprising a compound of formula I, as a free base or a pharmaceutically acceptable salt, solvate or solvate of salt thereof, for use in the prevention and/or treatment of conditions associated with glycogen synthase kinase-3.

The composition used in accordance with the present invention may be in a form suitable for oral administration, for example as a tablet, pill, syrup, powder, granule or capsule, for parenteral injection (including intravenous, subcutaneous, intramuscular, intravascular or infusion) as a sterile solution, suspension or emulsion, for topical administration as an ointment, patch or cream, for rectal administration as a suppository and for local administration in a body cavity or in a bone cavity.

Suitable daily doses of the compounds of the formula (I) used in the treatment of a mammal, including human, are approximately from 0.01 to 250 mg/kg bodyweight at peroral administration and from about 0.001 to 250 mg/kg bodyweight at parenteral administration. The typical daily dose of the active ingredients varies within a wide range and will depend on various factors such as the relevant indication, the route of administration, the age, weight and sex of the patient and may be determined by a physician.

For veterinary use the amounts of different components, the dosage form and the dose of the medicament may vary and will depend on various factors such as, for example the individual requirement of the animal treated.

A suitable pharmaceutically acceptable salt of the compound of formula (I) useful in accordance to the invention is, for example, an acid-addition salt, which is sufficiently basic, for example an inorganic or organic acid. In addition a suitable pharmaceutically acceptable salt of the compounds of the invention, which is sufficiently acidic, is an alkali metal salt, an alkaline earth metal salt or a salt with an organic base, which affords a physiologically-acceptable cation.

The dose required for the therapeutic or preventive treatment of a particular disease, disorder or a particular condition will necessarily be varied depending on the host treated, the route of administration and the severity of the illness or injury being treated.

In the context of the present specification, the term “therapy” also includes “prevention” unless there are specific indications to the contrary. The terms “therapeutic” and “therapeutically” should be construed accordingly.

In the context of the present specification, the term “disorder” also includes “condition” unless there are specific indications to the contrary.

### Medical Use

Surprisingly, it has been found that the compounds of formula (I) defined in the present invention, are well suited for inhibiting glycogen synthase kinase-3 (GSK3). Accordingly, said compound of the present invention is expected to be useful in the prevention and/or treatment of conditions associated with glycogen synthase kinase-3 activity, i.e. the compounds may be used to produce an inhibitory effect of GSK3 in mammals, including human, in need of such prevention and/or treatment.

GSK3 is highly expressed in the central and peripheral nervous system and in other tissues. Thus, it is expected that compound of the invention is well suited for the prevention and/or treatment of conditions associated with glycogen synthase kinase-3 in the central and peripheral nervous system. In particular, the compound of the invention is expected to be suitable for prevention and/or treatment of conditions associated with cognitive disorders and predemented states, especially dementia, Alzheimer's Disease (AD), Cognitive Deficit in Schizophrenia (CDS), Mild Cognitive Impairment (MC1), Age-Associated Memory Impairment (AAMI), Age-Related Cognitive Decline (ARCD) and Cognitive Impairement No Dementia (CIND), diseases associated with neurofibrillar tangle pathologies, Frontotemporal dementia (FTD), Frontotemporal dementia Parkinson's Type (FTDP), progressive supranuclear palsy (PSP), Pick's Disease, Niemann-Pick's Disease, corticobasal degeneration (CBD), traumatic brain injury (TBI) and dementia pugilistica.

One embodiment of the invention relates to the prevention and/or treatment of Alzheimer's Disease, especially the use in the delay of the disease progression of Alzheimer's Disease.

Other conditions are selected from the group consisting of Down's syndrome, vascular dementia, Parkinson's Disease (PD), postencephelatic parkinsonism, dementia with Lewy bodies, HIV dementia, Huntington's Disease, amyotrophic lateral sclerosis (ALS), motor neuron diseases (MND, Creurtfeld-Jacob's disease and prion diseases.

Other conditions are selected from the group consisting of attention deficit disorder (ADD), attention deficit hyperactivity disorder (ADHD) and affective disorders, wherein the affective disorders are Bipolar Disorder including acute mania, bipolar depression, bipolar maintenance, major depressive disorders (MDD) including depression, major depression, mood stabilization, schizoaffective disorders including schizophrenia, and dysthymia.

Other conditions are selected from the group consisting of Type I diabetes, Type II diabetes, diabetic neuropathy, alopecia, inflammatory diseases and cancer.

One embodiment of the invention relates to the use of a compound of the formula (I), as a free base or a pharmaceutically acceptable salt thereof, in the prevention and/or treatment of bone-related disorders or conditions in mammals.

One aspect of the invention is directed to the use of a compound of the formula (I), as a free base or a pharmaceutically acceptable salt thereof, to treat osteoporosis.

One aspect of the invention is directed to the use of a compound of the formula (I), as a free base or a pharmaceutically acceptable salt thereof, to increase and promote bone formation in mammals.

One aspect of the invention is directed to the use of a compound of the formula (I), as a free base or a pharmaceutically acceptable salt thereof, to increase bone mineral density in mammals.

Another aspect of the invention is directed to the use of a compound of the formula (I), as a free base or a pharmaceutically acceptable salt thereof, to reduce the rate of fracture and/or increase the rate of fracture healing in mammals.

Another aspect of the invention is directed to the use of a compound of the formula (I), as a free base or a pharmaceutically acceptable salt thereof, to increase cancellous bone formation and/or new bone formation in mammals.

Another aspect of the invention is directed to a method of prevention and/or treatment of bone-related disorders comprising administering to a mammal in need of such prevention and/or treatment, a therapeutically effective amount of a compound of the formula (I) as a free base or a pharmaceutically acceptable salt thereof.

Another aspect of the invention is directed to a method of prevention and/or treatment of osteoporosis comprising administering to a mammal in need of such prevention and/or treatment, a therapeutically effective amount of a compound of the formula (I) as a free base or a pharmaceutically acceptable salt thereof.

Another aspect of the invention is directed to a method of increasing bone formation comprising administering to a mammal in need of such treatment, a therapeutically effective amount of a compound of the formula (I) as a free base or a pharmaceutically acceptable salt thereof.

Another aspect of the invention is directed to a method of increasing bone mineral density comprising administering to a mammal in need of such treatment, a therapeutically effective amount of a compound of the formula (I) as a free base or a pharmaceutically acceptable salt thereof.

Another aspect of the invention is directed to a method of reducing the incidence of fracture comprising administering to a mammal in need of such treatment, a therapeutically effective amount of a compound of the formula (I) as a free base or a pharmaceutically acceptable salt thereof.

Another aspect of the invention is directed to a method of enhancing fracture healing comprising administering to a mammal in need of such treatment, a therapeutically effective amount of a compound of the formula (I) as a free base or a pharmaceutically acceptable salt thereof.

Another aspect of the invention is directed to said methods and wherein said mammal is a human.

Another aspect of the invention is directed to said methods and wherein said mammal is a vertibrate animal, preferably but not limited to bigger animals such as horses, camels, dromedars but not limited thereto.

The use of the GSK3 inhibitors, the compounds of formula (I), in primary and secondary ostopeorosis, where primary osteoporosis includes postmenopausal osteoporosis and senile osteoporosis in both men and women, and secondary osteoporosis includes cortison induced osteoporosis, as well as any other type of induced secondary osteoporosis, are included in the term osteoporosis. In addition to this, these GSK3 inhibitors may also be used in treatments of myeloma. These GSK3 inhibitors may be administered locally or systemically, in different formulation regimes, to treat these conditions.

The promotion and increasing of bone formation makes the compounds of the formula (I) suitable to reducing the incidence of fracture, to reduce the rate of fracture and/or increase the rate of fracture healing, to increase cancellous bone formation and/or new bone formation in mammals.

The use to promote and increase new bone formation may be in connection with surgery. This invention can be used during surgery, where the treating surgeon will place the invention locally in an appropriate formulation, near the deficient bone and/or in the body cavity. The bone may for instance have been broken, and utilizing the invention as described and claimed herein will then be placed in or near the fracture during open fracture repair. In some instances bone pieces may be missing (e.g. after tumour removal or severe casualties), and utilizing the invention as described and claimed herein will then be placed near the site of constructive bone surgery.

### Non-Medical Use

In addition to their use in therapeutic medicine, the compounds of formula I as a free base or a pharmaceutically acceptable salt thereof, are also useful as pharmacological tools in the development and standardisation of in vitro and in vivo test systems for the evaluation of the effects of inhibitors of GSK3 related activity in laboratory animals such as cats, dogs, rabbits, monkeys, rats and mice, as part of the search for new therapeutics agents.

### Pharmacology

### Determination of ATP Competition in Scintillation Proximit), GSK3β Assay.

### GSK3β Scintillation Proximity Assay.

The competition experiments were carried out in duplicate with 10 different concentrations of the inhibitors in clear-bottom microtiter plates (Wallac, Finland). A biotinylated peptide substrate, Biotin-Ala-Ala-Glu-Glu-Leu-Asp-Ser-Arg-Ala-Gly-Ser(PO₃H₂)-Pro-Gln-Leu (AstraZeneca, Lund), was added at a final concentration of 1 μM in an assay buffer containing 1 mU recombinant human GSK3β (Dundee University, UK), 12 mM morpholinepropanesulfonic acid (MOPS), pH 7.0, 0.3 mM EDTA, 0.01% β-mercaptorethanol, 0.004% Brij 35 (a natural detergent), 0.5% glycerol and 0.5 μg BSA/25 μl. The reaction was initiated by the addition of 0.04 μCi [γ-³³P]ATP (Amersham, UK) and unlabelled ATP at a final concentration of 1 μM and assay volume of 25 μl. After incubation for 20 minutes at room temperature, each reaction was terminated by the addition of 25 μl stop solution containing 5 mM EDTA, 50 μM ATP, 0.1% Triton X-100 and 0.25 mg streptavidin coated Scintillation Proximity Assay (SPA) beads (Amersham, UK). After 6 hours the radioactivity was determined in a liquid scintillation counter (1450 MicroBeta Trilux, Wallac). The inhibition curves were analysed by non-linear regression using GraphPad Prism, USA. The Kₘ value of ATP for GSK3β, used to calculate the inhibition constants (Kᵢ) of the various compounds, was 20 μM.

The following abbreviations have been used:

MOPS Morpholinepropanesulfonic acid
EDTA Ethylenediaminetetraacetic acid

### BSA Bovin Serum Albumin

### ATP Adenosine Triphosphate

### SPA Scintillation Proximity Assay

GSK3 Glycogen synthase kinase 3

### Results

Typical Kᵢ values for the compounds of the present invention are in the range of about 0.01 to about 10,000 nM. Other values for Kᵢ are in the range of about 0.01 to about 1000 nM. Further values for Kᵢ are in the range of about 0.01 nM to about 300 nM.

TABLE 1
Specimen result from assay.

| Example no | Kᵢ (nM) |
|---|---|
| 13 | 390 |
| 14 | 1400 |
| 21 | 779 |

1. A compound of formula I: 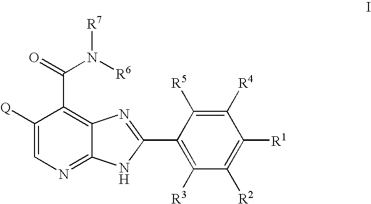
wherein;
Q is halogen;
R¹ is CH₂NR^{b}R^{c};
R², R³, R⁴ and R⁵ are independently selected from hydrogen and C₁₋₃alkyl;
R⁶ is hydrogen or C₁₋₆alkyl;
R⁷ is selected from hydrogen, C₁₋₆alkyl, C₁₋₆alkylaryl, aryl and heteroaryl, said C₁₋₆alkyl, C₁₋₆alkylaryl, aryl and heteroaryl are optionally substituted with one or more A;
A is halo, CN, OR^{a} or NR^{b}R^{c};
R^{a} is hydrogen, C₁₋₃alkyl or C₁₋₃haloalkyl, said C₁₋₃alkyl or C₁₋₃haloalkyl is optionally substituted with one or more C₁₋₃alkoxy;
R^{b} and R^{c} may, together with the atom to which they are attached, form a 4-, 5- or 6-membered heterocyclic ring containing one or more heteroatoms selected from N, O or S, wherein said heterocyclic ring is optionally substituted with one or more halo, C₁₋₃alkyl or C₁₋₃haloalkyl, and in which any sulphur atom is optionally oxidised to —SO₂—;
as a base or a pharmaceutically acceptable salt, solvate or solvate of a salt thereof.

2. A compound according to claim 1, wherein said halogen in Q is selected from bromo, chloro and fluoro.

3. A compound according to claim 1, wherein said R^{b}R^{c} together with the atom to which they are attached, form a 6-membered heterocyclic ring containing one or more heteroatoms selected from N or O.

4. A compound according to any one of claims 1 to 3, wherein said R², R³, R⁴ and R⁵ are hydrogen.

5. A compound according to any one of claims 1 to 3, wherein R⁶ is hydrogen and R⁷ is C₁₋₆alkyl, said C₁₋₆alkyl being optionally substituted with one or more A.

6. A compound according to claim 5, wherein A is OR^{a}.

7. A compound according to claim 6, wherein said R² in OR^{a} represents C₁₋₃alkyl.

8. A compound according to claim 1, wherein Q is halogen; R¹ is CH₂NR^{b}R^{c}, R^{b}R^{c} together with the atom to which they are attached, form a 4-, 5- or 6-membered heterocyclic ring containing one or more heteroatoms selected from N, O or S; R², R³, R⁴ and R⁵ are hydrogen; R⁶ is hydrogen and R⁷ is C₁₋₆alkyl, said C₁₋₆alkyl is substituted with one A, said A being OR^{a}, said R^{a} in OR^{a} representing C₁₋₃alkyl.

9. A compound according to claim 1, wherein Q is halogen; R¹ is CH₂NR^{b}R^{c}, R^{b}R^{c} together with the atom to which they are attached, form a 6-membered heterocyclic ring containing one or more heteroatoms selected from N, O or S; R², R³, R⁴ and R⁵ are hydrogen; R⁶ is hydrogen and R⁷ is C₁₋₆alkyl, said C₁₋₆alkyl is substituted with one A, said A being OR^{a}, wherein said R^{a} is C₁₋₃alkyl.

10. A compound according to claim 1, wherein Q is halogen; R¹ is CH₂NR^{b}R^{c}, R^{b}R^{c} together with the atom to which they are attached, form a morpholine; R², R³, R⁴ and R⁵ are hydrogen; R⁶ is hydrogen and R⁷ is C₁₋₆alkyl, said C₁₋₆alkyl is substituted with one A, said A being OR^{E}, wherein said R^{a} is C₁₋₃alkyl.

11. A compound according to claim 1, selected from:
6-Bromo-N-(3-methoxypropyl)-2-[4-(morpholin-4-ylmethyl)phenyl]-3H-imidazo[4,5-b]pyridine-7-carboxamide hydrochloride;
6-Chloro-N-(3-methoxypropyl)-2-[4-(morpholin-4-ylmethyl)phenyl]-3H-imidazo[4,5-b]pyridine-7-carboxamide hydrochloride;
6-Fluoro-N-(3-methoxypropyl)-2-[4-(morpholin-4-ylmethyl)phenyl]-3H-imidazo[4,5-b]pyridine-7-carboxamide;
as a base or an alternative pharmaceutically acceptable salt, solvate or solvate of a salt thereof.

12-14. (canceled)

15. A compound selected from:
Methyl 4-(6-bromo-3H-imidazo[4,5-b]pyridin-2-yl)benzoate;
Methyl 4-(6-chloro-3H-imidazo[4,5-b]pyridin-2-yl)benzoate;
Methyl 4-(6-bromo-7-chloro-3H-imidazo[4,5-b]pyridin-2-yl)benzoate;
Methyl 4-(6,7-dichloro-3H-imidazo[4,5-b]pyridin-2-yl)benzoate;
Methyl 4-(7-chloro-6-fluoro-3H-imidazo[4,5-b]pyridin-2-yl)benzoate;
Methyl 4-(6-bromo-7-iodo-3H-imidazo[4,5-b]pyridin-2-yl)benzoate;
Methyl 4-(6-chloro-7-iodo-3H-imidazo[4,5-b]pyridin-2-yl)benzoate;
Methyl 4-(6-fluoro-7-iodo-3H-imidazo[4,5-b]pyridin-2-yl)benzoate
4-(6-Bromo-7-iodo-3H-imidazo[4,5-b]pyridin-2-yl)benzoic acid;
4-(6-Chloro-7-iodo-3H-imidazo[4,5-b]pyridin-2-yl)benzoic acid;
4-(6-Fluoro-7-iodo-3H-imidazo[4,5-b]pyridin-2-yl)benzoic acid
6-Bromo-7-iodo-2-[4-(morpholin-4-ylcarbonyl)phenyl]-3H-imidazo[4,5-b]pyridine;
6-Chloro-7-iodo-2-[4-(morpholin-4-ylcarbonyl)phenyl]-3H-imidazo[4,5-b]pyridine;
6-Fluoro-7-iodo-2-[4-(morpholin-4-ylcarbonyl)phenyl]-3H-imidazo[4,5-b]pyridine
6-Bromo-7-iodo-2-[4-(morpholin-4-ylmethyl)phenyl]-3H-imidazo[4,5-b]pyridine;
6-Chloro-7-iodo-2-[4-(morpholin-4-ylmethyl)phenyl]-3H-imidazo[4,5-b]pyridine and
6-Fluoro-7-iodo-2-[4-(morpholin-4-ylmethyl)phenyl]-3H-imidazo[4,5-b]pyridine.

16. A compound according to any one of claim 4, wherein R⁶ is hydrogen and R⁷ is C₁₋₆alkyl, said C₁₋₆alkyl being optionally substituted with one or more A.

17. A pharmaceutical formulation comprising as active ingredient a therapeutically effective amount of a compound according to in claim 1 or claim 11 in association with pharmaceutically acceptable excipients, carriers or diluents.

18. A method of prevention and/or treatment of bone-related disorders including osteoporosis and increased bone formation, comprising administering to a human in need of such prevention and/or treatment a therapeutically effective amount of a compound as defined in claim 1 or claim 11.
